(19) United States
(12) Patent Application Publication
(10) Pub. No.: US 2025/0277791 A1
(43) Pub. Date: Sep. 4, 2025

(54) INFLUENCE OF RECEPTOR INTEGRATIVE ANALYSIS ON HR+/HER2+ BREAST CANCER MOLECULAR SUBTYPES AND PROGNOSIS

(71) Applicant: CANCER HOSPITAL CHINESE ACADEMY OF MEDICAL SCIENCE, Beijing (CN)

(72) Inventors: Peng YUAN, Beijing City (CN); Jie JU, Beijing City (CN); Feng DU, Beijing City (CN)

(21) Appl. No.: 18/842,768

(22) PCT Filed: Apr. 7, 2023
(86) PCT No.: PCT/CN2023/086898

§ 371 Date: Aug. 29, 2024

(30) Foreign Application Priority Data
Apr. 22, 2022 (CN) 202210431139.6

(51) Int. Cl. G01N 33/574; C12Q 1/6886

(52) U.S. Cl. CPC G01N 33/57415; C12Q 1/6886; C12Q 2600/112; C12Q 2600/118; C12Q 2600/158

(57) ABSTRACT

Provided in the present invention are a method for identifying HER2-enriched subtype breast cancer or HER2-enriched-like subtype breast cancer by means of measuring and comparing the expression level of ERBB2 mRNA and the expression level of ESR1 mRNA or by means of measuring and comparing the ratio of HER-positive cells and the ratio of ER-positive cells, a kit, an apparatus, and the use of a corresponding detection reagent for identifying HER2-enriched subtype breast cancer or HER2-enriched-like subtype breast cancer.

### RELATED APPLICATIONS

The present application is a U.S. National Phase of International Application Number PCT/CN2023/086898 filed Apr. 7, 2023, which claims priority to Chinese Application Number 202210431139.6 filed Apr. 22, 2022.

### TECHNICAL FIELD

The disclosure relates to the identification and prognosis of breast cancer molecular subtypes, and in particular to the identification of patients with HR+/HER2+ breast cancer.

### BACKGROUND

Hormone receptor-positive and human epidermal growth factor receptor 2-positive (HR+/HER2+) breast cancer accounts for about 5%-10% of all breast cancers. However, the lack of knowledge regarding the heterogeneity in HR+/HER2+ breast cancer remains a barrier to precise treatment.

Human epidermal growth factor receptor 2 (HER2) is overexpressed in about 20% of breast cancer cases [Guarneri V et al., (2010) Cancer Treat Rev 36 Suppl 3: S62-66. doi: 10.1016/S0305-7372(10)70022-0]. About 50% of HER2+ breast cancers also express a hormone receptor (HR) [Cancer Genome Atlas Network (2012), Nature 490: 61-70. doi: 10.1038/nature11412]. More treatment options are available for HR+/HER2+ patients than for HR−/HER2+ patients. Currently, the standard first-line treatment for metastatic HR+/HER2+ disease consists of chemotherapy and an anti-HER2 therapy [Slamon D J et al., (2001) NEngl J Med 344: 783-792. doi: 10.1056/NEJM200103153441101; Marty M et al., (2005) J Clin Oncol 23: 4265-4274. doi: 10.1200/JCO.2005.04.173]. Recently, several studies demonstrated that the combination of an endocrine therapy with an anti-HER2 therapy could also benefit some HR+/HER2+ patients with metastasis equally in the case that they were exempted from chemotherapy [Huober J et al., (2012) Breast 21: 27-33. doi: 10.1016/j.breast.2011.07.006; Tolaney S M et al., (2020) Lancet Oncol 21: 763-775. doi: 10.1016/S1470-2045(20)30112-1]. Results of the SYSUCC-002 study were reported at the American Society of Clinical Oncology (ASCO) annual meeting in 2021, which showed the non-inferiority of an endocrine therapy in combination with an anti-HER2 therapy as the first-line treatment for HR+/HER2+ metastatic breast cancer compared to chemotherapy in combination with an anti-HER2 therapy [Zhongyu Yuan J H et al., (2021) Journal of Clinical Oncology 39: 15_suppl:1003-1003. doi: 10.1186/1471-2407-12-602].

Although significant progress has been made, the influence of tumor heterogeneity on HR+/HER2+ breast cancer has been underestimated. Emerging evidence supports the influence of tumor heterogeneity on clinical outcomes and drug sensitivity. First, all four main PAM50 intrinsic subtypes are present in HR+/HER2+ diseases [Cejalvo J M et al., (2018) Cancer Treat Rev 67: 63-70. doi: 10.1016/j.ctrv.2018.04.015; Zhao S et al., (2019) Theranostics 9: 4935-4945. doi: 10.7150/thno.35730]. Patients with the liminal types are considered to have a better prognosis and stronger response to the endocrine therapy [Prat A et al., (2016) JAMA Oncol 2: 1287-1294. doi: 10.1001/jamaoncol.2016.0922; Ciruelos E et al., (2020) Clin Cancer Res 26: 5820-5829. doi: 10.1158/1078-0432.CCR-20-0844]. Patients with the HER2-enriched subtype are highly sensitive to the anti-HER2 therapy [Schettini F et al., (2020) Cancer Treat Rev 84: 101965. doi: 10.1016/j.ctrv.2020.101965; Prat A et al., (2020) J Natl Cancer Inst 112: 46-54. doi: 10.1093/jnci/djz042].

Furthermore, the heterogeneity in HR+/HER2+ breast cancer can be divided into inter-tumor heterogeneity and intra-tumor heterogeneity, and is enhanced by the diversity of ERBB2 and ESR1 expression. It is observed that in the immunohistochemically defined HER2+ group, the levels of HER2 are not homogeneous, and the levels of ERBB2 mRNA and protein increase progressively with immunohistochemistry scores [Griguolo G et al., (2020) Cancers (Basel) 12. doi: 10.3390/cancers12071902]. Furthermore, intra-tumor heterogeneity exists in different states of HER2 in different regions of the same tumor [Marchiò C et al., (2021) Semin Cancer Biol 72: 123-135. doi: 10.1016/j.semcancer.2020.02.016]. Importantly, these heterogeneities are significantly correlated with the efficacy of anti-HER2 therapy in both neoadjuvant and advanced patients. [Hou Y et al., (2017) Breast Cancer Res Treat 166: 447-457. doi: 10.1007/s10549-017-4453-8; Hurvitz S A et al., (2019) J Clin Oncol 37: 2206-2216. doi: 10.1200/JCO.19.00882].

At present, our understanding of the tumor heterogeneity in HR+/HER2+ breast cancer is relatively limited. The lack of feasible and reproducible indicators to describe tumor heterogeneity limits further clinical application of this concept. Therefore, there is a need for an indicator to easily and quickly describe and identify the heterogeneity in patients with HR+/HER2+ breast cancer.

### SUMMARY

The inventors found that a combined analysis with ERBB2 and ESR1 expression could contribute to identifying patients with specific subtypes of HR+/HER2+ breast cancer. The inventors describe the tumor heterogeneity in HR+/HER2+ breast cancer from multiple aspects by: 1) determining the distribution of PAM50 intrinsic subtypes, 2) comparing the DNA mutational profile and RNA expression features between HER2-enriched and non-HER2-enriched subtypes, and 3) showing the heterogeneity using multiplex immunofluorescence (mIF) to determine the states of HER2 and estrogen receptor (ER) simultaneously. We have established a novel indicator to identify the HER2-enriched subtype and/or the HER2-enriched-like subtype in patients with HR+/HER2+ breast cancer.

In one aspect, the disclosure provides a method for helping to predict whether a breast cancer in a subject is a breast cancer of an HER2-enriched subtype and/or a breast cancer of an HER2-enriched-like subtype with a poor prognosis, comprising:
measuring or obtaining the level of ERBB2 mRNA and the level of ESR1 mRNA in a biological sample from a subject and/or the number/proportion of HER2-positive cells and the number/proportion of ER-positive cells in the biological sample from the subject, and calculating the following indicators rH/E and/or prH/E,
rH/E=log₂(FPKM_{ERBB2}+1)/[log₂(FPKM_{ESR1}+1)+1], wherein FPKM_{ERBB2} denotes the level of ERBB2 mRNA expressed as fragments per kilobase of transcript per million mapped reads (FPKM), and FPKM_{ESR1} denotes the level of ESR1 mRNA expressed as FPKM,
prH/E=the proportion of HER2-positive cells×100/(the proportion of ER-positive cells×100+1), wherein the proportions of the cells are proportions relative to the total number of tumor cells.

Relative to analysis of the level of ERBB2 or ESR1 mRNA alone, rH/E is more able to distinguish between HER2-enriched and non-HER2-enriched subtypes, and the greater the rH/E (e.g., compared to rH/E for a known breast cancer of a non-HER2-enriched subtype), the more prone the subject to a breast cancer of an HER2-enriched subtype with a poor prognosis. In the indicator prH/E=the proportion of HER2-positive cells×100/(the proportion of ER-positive cells×100+1), the proportions of the cells are proportions relative to the total number of tumor cells (e.g., the proportions of the cells are proportions relative to the total number of tumor cells in a field of view, which are obtained by randomly selecting 10 high magnification fields of view (×400) in an mIF-stained section of each patient, counting, and averaging), and in the case of prH/E≥1.5, the breast cancer in the subject is indicated to be a breast cancer of an HER2-enriched-like subtype with a poor prognosis.

In one aspect, the disclosure provides use of a detection reagent for detecting ERBB2 mRNA and a detection reagent for detecting ESR1 mRNA and/or a detection reagent for detecting HER2-positive cells and a detection reagent for detecting ER-positive cells in the preparation of a kit for predicting a breast cancer of an HER2-enriched subtype and/or a breast cancer of an HER2-enriched-like subtype with a poor prognosis.

In one aspect, the disclosure provides a kit for predicting a breast cancer of an HER2-enriched subtype and/or a breast cancer of an HER2-enriched-like subtype with a poor prognosis, comprising a detection reagent for detecting ERBB2 mRNA and a detection reagent for detecting ESR1 mRNA and/or a detection reagent for detecting HER2-positive cells and a detection reagent for detecting ER-positive cells.

In one aspect, the disclosure provides a detection reagent for detecting ERBB2 mRNA and a detection reagent for detecting ESR1 mRNA and/or a detection reagent for detecting HER2-positive cells and a detection reagent for detecting ER-positive cells, for use in predicting a breast cancer of an HER2-enriched subtype and/or an HER2-enriched-like subtype with a poor prognosis.

In one aspect, the disclosure provides a method for treating a subject with breast cancer, comprising measuring or obtaining the number/proportion of HER2-positive cells and the number/proportion of ER-positive cells in a biological sample from the subject, and calculating an indicator prH/E=the proportion of HER2-positive cells×100/(the proportion of ER-positive cells×100+1), wherein the proportions of the cells are proportions relative to the total number of tumor cells (e.g., by randomly selecting 10 high magnification fields of view (×400) in an mIF-stained section of each patient, counting, and averaging), wherein in the case of prH/E≥1.5, the breast cancer is indicated to be a breast cancer of an HER2-enriched-like subtype with a poor prognosis, and an intensive anti-HER2 therapy may be considered to be administered to the subject.

In one aspect, the disclosure provides an apparatus for predicting a breast cancer of an HER2-enriched subtype and/or an HER2-enriched-like subtype with a poor prognosis, comprising:
a component for measuring or receiving the number/proportion of HER2-positive cells in a biological sample from a subject,
a component for measuring or receiving the number/proportion of ER-positive cells in the biological sample from the subject, and
a component for calculating an indicator prH/E, wherein prH/E=the proportion of HER2-positive cells×100/(the proportion of ER-positive cells×100+1), wherein the proportions of the cells are proportions relative to the total number of tumor cells (e.g., by randomly selecting 10 high magnification fields of view (×400) in an mIF-stained section of each patient, counting, and averaging);
optionally comprising a component for indicating that the subject has a breast cancer of an HER2-enriched-like subtype with a poor prognosis in the case of prH/E≥1.5.

### BRIEF DESCRIPTION OF THE DRAWINGS

FIGS. 1A and 1B: distribution of PAM50 intrinsic subtypes of patients with HR+/HER2+ breast cancer in TCGA cohort (FIG. 1A) and METABRIC cohort (FIG. 1B).

FIGS. 2A and 2B: molecular characteristics of HER2-enriched and non-HER2-enriched subtypes of HR+/HER2+ breast cancer in the TCGA cohort. FIG. 2A: a waterfall plot of hot somatic mutations in HER2-enriched subtype and non-HER2-enriched subtype in the TCGA cohort shows that the mutation rates of TP53 and ERBB3 in patients with the HER2-enriched subtype are significantly higher than those in patients with the non-HER2-enriched subtype (48% vs. 24%, P<0.01; 15% vs. 1%, P<0.001), whereas the mutation rate of PIK3CA in patients with HER2-enriched ER+HER2+ breast cancer is significantly lower than that in patients with non-HER2-enriched breast cancer (15% vs. 42%, P<0.001); FIG. 2B: a heatmap of differentially expressed genes for HER2-enriched subtype and non-HER2-enriched subtype in the TCGA cohort shows that the HER2-enriched subtype highly expresses genes related to a G2/M checkpoint, an E2F transcription factor, and an mTOR complex 1 signaling pathway, but lowly expresses genes related to epithelial mesenchymal transition (EMT), ER, and a tumor necrosis factor-α (TNF-α) signaling pathway mediated by NF-κB. TCGA: The Cancer Genome Atlas; mTORCL: mechanistic target of rapamycin complex 1; TNF-α: tumor necrosis factor-α; EMT: epithelial mesenchymal transition; HER2-E: HER2-enriched subtype; non-HER2-E: non-HER2-enriched subtype; ***p<0.001; **p<0.01.

FIGS. 3A-3F: Combined analysis of ERBB2 and ESR1 is a better marker for predicting the HER2-enriched subtype. FIG. 3A: the expression level of ERBB2 in different PAM50 intrinsic subtypes in the TCGA cohort; FIG. 3B: the expression level of ESR1 in different PAM50 intrinsic subtypes in the TCGA cohort; FIG. 3C: ROC curves of different markers for the discrimination of the HER2-enriched subtype and the non-HER2-enriched subtype in the TCGA cohort; FIG. 3D: the expression level of ERBB2 in different PAM50 intrinsic subtypes in the METABRIC cohort; FIG. 3E: the expression level of ESR1 in different PAM50 intrinsic subtypes in the METABRIC cohort; FIG. 3F: ROC curves of different markers of for the discrimination of the HER2-enriched subtype and the non-HER2-enriched subtype in the METABRIC cohort. TCGA: The Cancer Genome Atlas; METABRIC: Molecular Taxonomy of Breast Cancer International Consortium; AUC: area under the curve; ROC: receiver operating characteristic; HER2-E: HER2-enriched subtype.

FIGS. 4A-4H: Multiplex immunofluorescence shows 4 different types of tumor cells in a patient with HR+/HER2+ breast cancer in the CAMS cohort. IHC results: ER=80%, PR=10%, and HER2=3+. mIF results: ER+HER2+ cells=64.4%, ER+HER2− cells=5.2%, ER-HER2+ cells=16.4%, and ER-HER2− cells=14%. FIG. 4A: H&E image. FIG. 4B: ER IHC image. FIG. 4C: PR IHC image. FIG. 4D: HER2 IHC image. FIG. 4E: cell nucleus IF image. FIG. 4F: HER2 IF image. FIG. 4G: ER IF image. FIG. 4H: HER2 and ER mIF image (1: ER+HER2− (blue cell nucleus, red fluorescence visible in cell nucleus, no green fluorescence on cell membrane under microscope); 2: ER-HER2+(blue cell nucleus, no red fluorescence in cell nucleus, and green fluorescence visible on cell membrane under microscope); 3: ER-HER2− (blue cell nucleus, no red fluorescence in cell nucleus, and no green fluorescence on cell membrane under microscope); 4: ER+HER2+(blue cell nucleus, red fluorescence visible in cell nucleus, and green fluorescence visible on cell membrane under microscope)). H&E: hematoxylin-eosin staining; IHC: immunohistochemistry; IF: immunofluorescence; HER2: human epidermal growth factor receptor 2; ER: estrogen receptor; PR: progestogen receptor.

FIGS. 5A-5C: 4 types of tumor cells reflect intra-tumor heterogeneity in HR+/HER2+ breast cancer. FIG. 5A: the distribution of 4 types of tumor cells in each patient in the CAMS cohort;

FIG. 5B: the proportion of patients containing 2, 3, or 4 types of tumor cells; FIG. 5C: Spearman's correlation analysis between 4 types of tumor cells. **** p<0.0001; ***p<0.001; **p<0.01; *p<0.05; NS: not shown; HER2: human epidermal growth factor receptor 2; and ER: estrogen receptor.

FIG. 6: Patients with the HER2-enriched subtype show a significantly reduced disease-free survival rate compared to patients with the non-HER2-enriched subtype.

### DETAILED DESCRIPTION

Although various embodiments and aspects of the disclosure have been shown and described herein, it will be obvious to those skilled in the art that these embodiments and aspects are merely illustrative of the disclosure. Numerous variations, changes, and substitutions may be made by those skilled in the art without departing from the spirit of the disclosure. It should be understood that various alternatives to the embodiments of the disclosure described herein may be employed in implementing the disclosure.

Unless otherwise defined, all technical and scientific terms used herein have the same meaning as commonly understood by those skilled in the art to which the disclosure belongs. The techniques and methods described herein are generally performed according to methods well known in the art and described in the references cited in this specification, see, e.g., Sambrook et al. Molecular Cloning: A Laboratory Manual (3rd ed., Cold Spring Harbor Laboratory Press, Cold Spring Harbor, N. Y. (2001)). All references cited herein, including patents, patent applications, articles, textbooks, and the like, and references cited therein, are incorporated herein by reference in their entirety.

The existence of all 4 main PAM50 intrinsic molecular subtypes clearly demonstrates significant inter-tumor heterogeneity in HR+/HER2+ breast cancer. The distribution of these 4 subtypes that have been reported varies across studies. Consistent with some studies [Carey L A et al., (2016) J Clin Oncol 34: 542-549. doi: 10.1200/JCO.2015.62.1268], the disclosure shows that: the liminal B subtype accounts for the highest proportion, whereas the proportions of HER2-enriched and liminal A subtype are similar. In contrast, other studies have reported that the HER2-enriched subtype is predominant in HR+/HER2+ tumors [Tolaney S M et al., (2019) J Clin Oncol 37: 1868-1875. doi: 10.1200/JCO.19.00066; Llombart-Cussac A et al., (2017) Lancet Oncol 18: 545-554. doi: 10.1016/S1470-2045(17) 30021-9]. the disclosure demonstrates the existence of considerable inter-tumor and intra-tumor heterogeneities in HR+/HER2+ breast cancer. We observed significant differences in DNA mutations and gene expression profiles between HER2-enriched and non-HER2-enriched subtypes. In addition to ERBB2 expression, the diversity of ESR1 expression also affects tumor heterogeneity. The relative expression of ERBB2 to ESR1 may contribute to identifying patients with the HER2-enriched subtype. Combined analysis of ERBB2 and ESR1 expressions may provide a simpler and more cost-effective method for identifying patients with specific subtypes in this group. The disclosure found that the level of ERBB2 expression relative to ESR1 could predict the HER2-enriched subtype more accurately than ERBB2 expression alone.

The disclosure provides an indicator rH/E for predicting a breast cancer of an HER2-enriched subtype with a poor prognosis, which is calculated as follows: rH/E=log₂(FPKM_{ERBB2}+1)/[log₂(FPKM_{ESR1}+1)+1], wherein FPKM_{ERBB2} denotes the level of ERBB2 mRNA expressed as fragments per kilobase of transcript per million mapped reads (FPKM), and FPKM_{ESR1} denotes the level of ESR1 mRNA expressed as FPKM. The greater the rH/E (e.g., compared to rH/E for a known breast cancer of the non-HER2-enriched subtype), the more prone the subject to a breast cancer of an HER2-enriched subtype with a poor prognosis. The disclosure provides an indicator prH/E for predicting a breast cancer of an HER2-enriched-like subtype with a poor prognosis, which is calculated as follows: prH/E=the proportion of HER2-positive cells×100/(the proportion of ER-positive cells×100+1), wherein the proportions of the cells are proportions relative to the total number of tumor cells. In the case of prH/E≥1.5, the breast cancer is an HER2-enriched-like subtype with a poor prognosis. Accordingly, an intensive anti-HER2 therapy can be administered to patients having the described breast cancer of the HER2-enriched-like subtype with a poor prognosis for the treatment of breast cancer.

In one aspect, the disclosure provides a method for predicting whether a breast cancer in a subject is a breast cancer of an HER2-enriched subtype with a poor prognosis, comprising: measuring or obtaining the level of ERBB2 mRNA and the level of ESR1 mRNA in a biological sample from the subject, and
calculating an indicator rH/E=log₂(FPKM_{ERBB2}+1)/[log₂(FPKM_{ESR1}+1)+1], wherein FPKM_{ERBB2} denotes the level of ERBB2 mRNA expressed as FPKM and FPKM_{ESR1} denotes the level of ESR1 mRNA expressed as FPKM, and the greater the rH/E (e.g., compared to rH/E for a known breast cancer of a non-HER2-enriched subtype), the more prone the subject to a breast cancer of an HER2-enriched subtype with a poor prognosis.

In one aspect, the disclosure provides a method for predicting whether a breast cancer in a subject is a breast cancer of an HER2-enriched-like subtype with a poor prognosis, comprising:
measuring or obtaining the number/proportion of HER2-positive cells and the number/proportion of ER-positive cells in a biological sample from the subject, optionally measuring or obtaining the total number of tumor cells (e.g., an average of 10 fields of view) in the biological sample, wherein the proportions are proportions relative to the total number of tumor cells, and
calculating an indicator prH/E=the proportion of HER2-positive cells×100/(the proportion of ER-positive cells×100+1), wherein in the case of prH/E≥1.5, the breast cancer in the subject is indicated to be a breast cancer of an HER2-enriched-like subtype with a poor prognosis.

The scholars such as Perou and Sorlie divided breast cancer into 5 different subtypes at a molecular level using gene detection technology (the former was through a multi-level cluster analysis of microarray gene expression data, and the latter was through a gene expression cDNA microarray), including luminal A subtype, luminal B subtype, HER2-enriched subtype, basal cell-like subtype, and normal breast-like subtype (Perou C M et al. Molecular portraits of human breast tumours[J]. Nature, 2000, 406(6797):747-752. DOI:10.1038/35021093). As used herein, the term “breast cancer of an HER2-enriched subtype” refers to a breast cancer subtype that is divided based on gene expression differences by high-throughput next generation sequencing, which is characterized by a high frequency of ERBB2 amplification (about >80%), TP53 mutations (about 72%), PIK3CA mutations (about 39%), and cyclin D1 amplification (about 38%), and a lower frequency of PIK3R1 mutations (about 4%).

As used herein, the term “breast cancer of an HER2-enriched-like subtype” refers to a breast cancer subtype according to the indicator prH/E≥1.5 of the disclosure. The breast cancer of the HER2-enriched-like subtype has similar characteristics with the breast cancer of the HER2-enriched subtype, and is mainly driven by HER2.

As used herein, the term “ERBB2” refers to the protooncogene located on chromosome 17 (GenBank Accession Number: NM_004448), which encodes human epidermal growth factor receptor 2 (HER2), is involved in the activation of the proliferative pathway, and affects differentiation, invasion, and survival.

As used herein, the term “ESR1” refers to a gene located on chromosome 6 (GenBank Accession Number: NM_000125), which encodes an estrogen receptor (ER). ESR1 is an important marker and a driver of the Liminal subtype, which is highly expressed in some patients with HR+/HER2+ diseases. However, the effect of ESR1 expression on tumor heterogeneity is unclear.

As used herein, the term “HER2” refers to human epidermal growth factor receptor 2 (GenBank Accession Number: NP_004439), which is a product encoded by ERBB2 (GenBank Accession Number: NM_004448.4). HER2 is a 185 kD transmembrane protamine, abbreviated as p185, which consists of 1255 amino acids, with positions 720-987 belonging to the tyrosine kinase domain. The HER2 protein is a transmembrane protein with tyrosine protein kinase activity and is one of the members of the EGFR family. The protein consists of an extracellular ligand binding region, a single-chain transmembrane region, and an intracellular protein tyrosine kinase region, with no known ligand. The HER2 protein mainly exerts its physiological effects by forming a homodimer or a heterodimer with family members including EGFR (HER1/erbB1), HER3/erbB3, and HER4/erbB4, which changes its conformation upon dimerization and activates intracellular tyrosine kinase activity, which in turn activates a downstream pathway. The signal transduction pathway mediated by the HER2 protein mainly includes an Ras/Raf/mitogen activated protein kinase (MAPK) pathway, a phosphatidylinositol 3 hydroxykinase (PI3K)/Akt pathway, a signal transducer and activator of transcription (STAT) pathway, a PLC pathway, and the like. The intensity and location of HER2 expression, of primary interest in previous studies, have a significant prognostic and predictive role [Marchiò C et al., (2021) Semin Cancer Biol 72: 123-135. doi: 10.1016/j.semcancer.2020.02.016; Hou Y et al., (2017) Breast Cancer Res Treat 166: 447-457. doi: 10.1007/s10549-017-4453-8; Baselga J et al., (2016) Clin Cancer Res 22: 3755-3763. doi: 10.1158/1078-0432.CCR-15-2499; Perez E A et al., (2019) BMC Cancer 19: 517. doi: 10.1186/s12885-019-5687-0].

As used herein, the term “ER” refers to the major type of estrogen receptor, ERα (GenBank Accession Number: NP_000116), which is localized to the cell nucleus. When estradiol binds to its ligand binding region, it can result in a conformational change in the ER, which recruits co-regulatory proteins to regulate gene transcription, thereby promoting the growth, proliferation and survival of tumor cells. In one embodiment, the ER is a human ER.

As used herein, FPKM is a commonly used value describing gene expression in RNA-seq technology, which is calculated as follows: FPKM_{gene}=all reads mapped to exons of gene A for a certain sample/(sum of all reads (in millions) for the certain sample×sum of lengths of the exons of gene A (kB)). The RPKM for a gene in a sample is equal to the ratio of the total number of reads falling on the gene (total exon reads) to the product of the total number of reads for the sample (mapped reads (in millions)) and the length of the gene (exon length (kB)).

It is within the ability of one skilled in the art to determine the FPKM value for a particular gene in a sample, and methods and tools for determining FPKM are known in the art, including, for example, but not limited to, polymerase chain reaction (PCR), next generation sequencing (NGS), and the like.

In one embodiment, the level of ERBB2 mRNA or the level of ESR1 mRNA, expressed as FPKM, is determined using any suitable detection reagent, e.g., a primer, a probe, and the like. In one embodiment, the level of ERBB2 mRNA or the level of ESR1 mRNA is determined using a primer.

As used herein, HER2-positive cells refer to cells having detectable expression of HER2 protein, which can be detected by any method known in the art, e.g., immunohistochemistry, fluorescence, radioactivity, luminescence, chemistry, enzymatic labeling, in situ hybridization, an assay based on other detection reagents that specifically bind to the HER2 protein expressed by the cells, or the like.

In one embodiment, a method for detecting HER2-positive cells may comprise the following steps: contacting a biological sample with a first molecule (e.g., an antibody) that specifically binds to an HER2 protein, wherein optionally, the first molecule is labeled, e.g., fluorescently labeled, radiolabeled, luminescent labeled, or enzyme-labeled; and then measuring the number/proportion of HER2-positive cells.

In one embodiment, a method for detecting HER2-positive cells may comprise the following steps: contacting a biological sample with a first molecule (e.g., a primary antibody) that specifically binds to an HER2 protein; contacting a complex of the HER2 protein and the first molecule with a second molecule (e.g., a secondary antibody, such as an anti-IgG antibody) that specifically binds to the HER2 protein, the first molecule, or the complex, wherein optionally, the second molecule is labeled, e.g., fluorescently labeled, radiolabeled, luminescent labeled, or enzyme-labeled; and then measuring the number/proportion of HER2-positive cells.

As used herein, ER-positive cells refer to cells having detectable expression of ER protein, which can be detected by any method known in the art, e.g., immunohistochemistry, fluorescence, radioactivity, luminescence, chemistry, enzymatic labeling, in situ hybridization, an assay based on other detection reagents that specifically bind to the ER protein expressed by the cells, or the like.

In one embodiment, the method for detecting ER-positive cells may comprise the following steps: contacting a biological sample with a third molecule (e.g., an antibody) that specifically binds to an ER protein, wherein the third molecule is labeled, e.g., fluorescently labeled, radiolabeled, luminescent labeled, or enzyme-labeled; and then measuring the number/proportion of ER-positive cells.

In one embodiment, the method for detecting ER-positive cells may comprise the following steps: contacting a biological sample with a third molecule (e.g., a primary antibody) that specifically binds to an ER protein; contacting a complex of the ER protein and the third molecule with a fourth molecule (e.g., a secondary antibody, such as an anti-IgG antibody) that specifically binds to the ER protein, the third molecule, or the complex, wherein optionally, the fourth molecule is labeled, e.g., fluorescently labeled, radiolabeled, luminescent labeled, or enzyme-labeled; and then measuring the number/proportion of ER-positive cells.

In one embodiment, HER2-positive cells or ER-positive cells may be determined as follows: tissues or cells obtained from a biological sample of a subject are contacted with a fluorescently labeled antibody (e.g., a different fluorescently labeled HER2 antibody or ER antibody), and then the number/proportion of HER2-positive cells or the number/proportion of ER-positive cells are simultaneously observed by microscopy.

The term “antibody”, “antigen-binding fragment”, or “immunogenic portion” as used herein has the meanings commonly known to those skilled in the art. “Antibody” refers to an immunoglobulin molecule that generally consists of two pairs of polypeptide chains, each pair having one light chain (LC) and one heavy chain (HC). The antibody may be a polyclonal antibody, a monoclonal antibody, a chimeric antibody, a humanized antibody, or a human antibody, and may be a labeled antibody and a fragment, a variant, or a derivative of the antibody. The antibody label may be a radioactive label, a fluorescent label, an enzymatic label, a chemiluminescent label, or a biotin group label. “Antigen-binding fragment” refers to a polypeptide fragment produced, for example, by recombinant DNA techniques or by enzymatic or chemical cleavage of an intact antibody, which retains the ability to specifically bind to the same antigen to which the full-length antibody binds and/or competes with the full-length antibody for specific binding to the antigen, and is also referred to as an “antigen-binding portion”. See Fundamental Immunology, Ch. 7 (Paul, W., ed., 2nd edition, Raven Press, N.Y. (1989), which is incorporated herein by reference in its entirety. Non-limiting examples of antigen-binding fragments include Fab, Fab′, F(ab′)₂, Fd, Fv, dAb, and complementarity determining region (CDR) fragments, single-chain antibodies (e.g., scFv), chimeric antibodies, diabodies, linear antibodies, nanobodies (e.g., technology from Ablynx), domain antibodies (e.g., technology from Domantis), and polypeptides that comprise at least a portion of an antibody sufficient to have antigen-specific binding ability. Antibodies or fragments thereof and preparation and use thereof are well known. Techniques for the preparation of polyclonal or monoclonal antibodies, ScFv fragments, and human or humanized antibodies are described, for example, in the following documents: Harlow et al., Antibodies: A Laboratory Manual, CSH Press, 1988; Ward et al., Nature 341 (1989) 544; Bird et al., Science 242 (1988) 423; Harlow, E. and Lane, D., Antibodies: A Laboratory Manual, Cold Spring Harbor Laboratory Press, Cold Spring Harbor, New York, 1999; WO94/02602; U.S. Pat. Nos. 5,223,409; 5,877,293; and WO93/01288.

As used herein, the term “specific binding” refers to a non-random binding reaction between two molecules, such as a reaction between an antibody and an antigen it targets. With reference to the interaction of an antibody with its binding partner (e.g., an antigen), the term “specific binding” or “specific recognition” as used herein means that the interaction is dependent on the existence of a particular amino acid sequence or structure (e.g., an antigenic determinant or epitope) on the binding partner. In other words, the antibody preferentially binds to or recognizes the binding partner even if the binding partner is present in a mixture of other molecules or organisms. The binding may be mediated by covalent or non-covalent interaction or a combination of both. In other words, the term “specific binding” or “specific recognition” means that an antibody is specifically immunoreactive with an antigenic determinant or epitope, but not with other antigenic determinants or epitopes. An antibody with specific or immunospecific binding to an antigen can bind with low affinity to other peptides or polypeptides as determined by, for example, radioimmunoassay (“RIA”), enzyme-linked immunosorbent assay (“ELISA”), BIACORE, or other assays known in the art. An antibody or fragment thereof with specific binding to an antigen can cross-react with a related antigen carrying the same epitope. Preferably, the antibody or fragment thereof with specific binding to the antigen does not cross-react with other antigens.

The strength or affinity of a specific binding interaction can be expressed in terms of the dissociation equilibrium constant (KD) of the interaction. The term “KD” refers to a dissociation equilibrium constant for a specific antibody-antigen interaction, which is used to describe the binding affinity between the antibody and the antigen. A smaller dissociation equilibrium constant indicates a stronger antibody-antigen binding and a higher affinity between the antibody and the antigen. In some embodiments, an antibody that specifically binds to a certain antigen (or an antibody that is specific for a certain antigen) means that the antibody binds to the antigen with an affinity (KD) less than about 10⁻⁸ M, e.g., less than about 10⁻⁸ M, 10⁻⁹ M, 10⁻¹⁰ M, or 10⁻¹¹ M, or less. In certain embodiments, when KD is ≤10×10⁻⁸ M, the antibody or the antigen-binding fragment thereof of the disclosure is considered to specifically bind to the HER2 or ER protein. Antibodies specific for the HER2 protein and for the ER protein are available to those skilled in the art.

As used herein, the proportion of HER2-positive cells and the proportion of ER-positive cells refer to proportions relative to the total number of tumor cells, e.g., the proportion of HER2-positive cells and the proportion of ER-positive cells relative to the total number of tumor cells in one counting region (e.g., one tissue section or microscopic field of view) or a mean of the total numbers of tumor cells in a plurality of counting regions (e.g., a plurality of, e.g., 2, 3, 4, 5, 6, 7, 8, 9, 10 or more, tissue sections or microscopic fields of view). In one embodiment, the proportion is a mean in a plurality of samples or regions, e.g., 10 high magnification fields of view (×400 times).

The total number of tumor cells can be determined by any suitable method known in the art. In one embodiment, the total number of tumor cells in a biological sample can be determined, for example, by a hemocytometer method, by staining, such as with trypan blue, and the like. “Subject” as used herein refers to any organism. In some embodiments, the subject is an animal, non-limiting examples of which include humans, other mammals such as cows, rats, mice, dogs, monkeys, goats, sheep, cows, deer, and other non-mammals. In certain embodiments, the subject is a human, preferably a medical subject, and more preferably a cancer patient. In one embodiment, the subject has breast cancer.

As used herein, the “biological sample” is any biological sample from a subject to be tested, in particular a sample comprising a nucleic acid or polypeptide, such as a tissue or organ, and in particular a tumor tissue sample. The sample for detection in the method of the disclosure should generally be collected in a clinically acceptable manner, for example in a manner that protects the nucleic acid or protein. The sample may also be pretreated to increase accessibility of the target molecule, such as by lysis (mechanical lysis, chemical lysis, enzymatic lysis, etc.), purification, centrifugation, separation, and the like. The sample may also be labeled to facilitate detection of the existence of the target molecule (fluorescent label, radioactive label, luminescent label, chemical label, enzymatic label, etc.). As used herein, the term “sample” also encompasses a tissue and/or cells and/or bodily fluid of a subject that has been taken from the subject and, for example, has been placed on a microscope slide, on which the claimed method is performed.

In one embodiment, the biological sample is a breast cancer tissue sample, e.g., a breast cancer tissue section.

In one embodiment, the method comprises the following steps:
measuring or obtaining the total number of tumor cells on a breast cancer tissue section, measuring or obtaining the number of HER2-positive cells and the number of ER-positive cells on the section, for example, by immunofluorescence, in particular a multiplex immunofluorescence method, and

calculating prH/E=the proportion of HER2-positive cells×100/(the proportion of ER-positive cells×100+1), wherein the proportions of the cells are proportions relative to the total number of tumor cells.

In one embodiment, the number of HER2-positive cells and the number of ER-positive cells are determined by a multiplex immunofluorescence method, e.g., using an HER2 antibody and an ER antibody.

In one embodiment, the measuring HER2-positive cells and the measuring ER-positive cells are performed on the same section of the sample. For example, a section is incubated with a primary antibody directed against one of the HER2 and ER proteins to bind to a target protein, then with a secondary antibody (e.g., labeled, such as enzyme-labeled, in particular horseradish peroxidase-labeled), and then positive cells are detected by appropriate means (e.g., detection of a signal emitted by the label, or addition of an enzyme substrate and detection of the corresponding product or color development); then, the section is incubated with another primary antibody directed against the other of the HER2 and ER proteins to bind to a target protein, then with a secondary antibody (e.g., labeled, such as enzyme-labeled, in particular horseradish peroxidase-labeled), and then positive cells are detected by appropriate means (e.g., detection of a signal emitted by the label, or addition of an enzyme substrate and detection of the corresponding product or color development).

Preferably, measuring the number/proportion of positive cells comprises analyzing at least one image, e.g., a digital image, of the same section in the first determination and the second determination, or images, e.g., digital images, of the sections in the first determination and the section of the second determination using computer-implemented image analysis techniques.

The quantification can be performed in a substantially automated, in particular more objective, manner by means of computer-implemented image analysis techniques.

In one embodiment, the method comprises adding an anti-HER2 primary antibody to a breast cancer tissue section and performing incubation, then adding an HRP-conjugated secondary antibody to the section and performing incubation, and adding a first chromogenic solution (e.g., FITC 488, Thermo) to the section and performing incubation; after antigen retrieval, adding an anti-ER primary antibody to the section and performing incubation, then adding an HRP-conjugated secondary antibody to the section and performing incubation, and adding a second chromogenic solution (e.g., Alexa 594, Thermo) to the section and performing incubation; after antigen retrieval, sealing the section (e.g., with a neutral resin containing DAPI), and finally, observing staining.

In one embodiment, the method comprises adding an anti-ER primary antibody to a breast cancer tissue section and performing incubation, then adding an HRP-conjugated secondary antibody to the section and performing incubation, and adding a first chromogenic solution (e.g., Alexa 594, Thermo) to the section and performing incubation; after antigen retrieval, adding an anti-HER2 primary antibody to the section and performing incubation, then adding an HRP-conjugated secondary antibody to the section and performing incubation, and adding a second chromogenic solution (e.g., FITC 488, Thermo) to the section and performing incubation; after antigen retrieval, sealing the section (e.g., with a neutral resin containing DAPI), and finally, observing staining.

The first chromogenic solution and the second chromogenic solution may be any suitable staining solution as long as their results for color development can result in the discrimination between the HER2 and ER proteins. The staining of the section can be observed using any method known in the art, such as using a confocal microscope.

The novel markers rH/E and prH/E have potential clinical applications. There is a strong correlation between molecular subtyping and disease prognosis and drug sensitivity. Patients with the liminal subtype have better outcomes than those with the non-liminal subtype [Prat A et al., (2016) JAMA Oncol 2: 1287-1294. doi: 10.1001/jamaoncol.2016.0922]. In addition, HER2-enriched tumors are the most sensitive to anti-HER2-based therapy [Schettini F et al., (2020) Cancer Treat Rev 84: 101965. doi: 10.1016/j.ctrv.2020.101965]. However, since gene testing has not become a routine diagnostic method, it is clinically valuable to develop technically arbitrary and cost-effective methods to identify specific subgroups of patients. In the disclosure, based on the AUC value of the ROC curve, rH/E and prH/E are more able to distinguish between HER2-enriched and non-HER2-enriched subtypes relative to analysis of ERBB2 or ESR1 expression alone. HER2 and ER were detected by mIF in 43 HR+/HER2+ patients from CAMS and an HER2-enriched-like subgroup was identified. Patients in this subgroup have a poorer prognosis, suggesting that more potent anti-HER2 therapy may be required to treat these patients with the HER2-enriched subtype and/or HER2-enriched-like subtype.

In one aspect, the disclosure provides use of a detection reagent for detecting HER2-positive cells and a detection reagent for detecting ER-positive cells in the preparation of a kit for predicting a breast cancer of an HER2-enriched-like subtype of with a poor prognosis. In one aspect, the disclosure provides use of a detection reagent for detecting HER2-positive cells and a detection reagent for detecting ER-positive cells for predicting a breast cancer of an HER2-enriched-like subtype with a poor prognosis.

In one aspect, the disclosure provides use of a detection reagent for detecting the level of ERBB2 mRNA expressed as FPKM and a detection reagent for detecting the level of ESR1 mRNA expressed as FPKM in the preparation of a kit for predicting a breast cancer of an HER2-enriched subtype with a poor prognosis. In one aspect, the disclosure provides use of a detection reagent for detecting the level of ERBB2 mRNA expressed as FPKM and a detection reagent for detecting the level of ESR1 mRNA expressed as FPKM for predicting a breast cancer of an HER2-enriched subtype with a poor prognosis.

As used herein, the “detection reagent for detecting the level of ERBB2 mRNA expressed as FPKM” and “detection reagent for detecting the level of ESR1 mRNA expressed as FPKM” are any suitable molecule or compound capable of detecting and quantifying the level of ERBB2 mRNA or the level of ESR1 mRNA expressed as FPKM, for example, by Northern blotting, by selective hybridization, or using a substrate, such as a nucleic acid molecule array, a DNA chip, a primer, and a probe, coated with an oligonucleotide probe. In one embodiment, the detection reagent capable of detecting and quantifying the level of ERBB2 mRNA or the level of ESR1 mRNA is a primer.

In one embodiment, the kit may further comprise instructions indicating the calculation of rH/E=log₂(FPKM_{ERBB2}+1)/[log₂(FPKM_{ESR1}+1)+1], wherein the greater the rH/E (e.g., compared to rH/E for a known breast cancer of a non-HER2-enriched subtype), the more prone the subject to a breast cancer of an HER2-enriched subtype with a poor prognosis.

As used herein, the terms “detection reagent for detecting HER2-positive cells” and “detection reagent for detecting ER-positive cells” refer to molecules or compounds (e.g., antibodies of the HER2 protein and the ER protein or conjugates thereof) capable of detecting HER2-positive cells and ER-positive cells, and can include, for example, but are not limited to, polypeptides, nucleic acids, carbohydrates, lipids, small molecular weight compounds, oligonucleotides, oligopeptides, RNA interference (RNAi), antisense RNA, recombinant proteins, antibodies or conjugates thereof, ligands, aptamers, primers, probes, fusion proteins, and the like.

In one embodiment, the detection reagent is a molecule or compound that specifically binds to the HER2 protein or the ER protein, which may be labeled, e.g., fluorescently labeled, radiolabeled, luminescent labeled, or enzyme-labeled, so that cells bound by the detection reagent can be detected.

In one embodiment, the detection reagent is an antibody or an antigen-binding fragment thereof for the HER2 protein and the ER protein. For example, antibodies against the HER2 protein that may be used in the disclosure include, but are not limited to, HER2/ErbB2 (29D8) rabbit antibodies, HER2/ErbB2 (44E7) mouse antibodies, and HER2/ErbB2 (D8F12) rabbit antibodies, and antibodies against the ER protein include, but are not limited to, estrogen receptor a (D6R2W) rabbit antibodies and estrogen receptor a (D8H8) rabbit antibodies.

HER2-positive cells and ER-positive cells in a sample may be revealed or analyzed using any technique known to the skilled person, e.g., using antibodies specific for the HER2 or ER protein or fragments or derivatives of the antibodies, preferably antibodies specific for the HER2 or ER protein or fragments (such as Fab, Fab′, and CDR) of such antibodies or derivatives (such as single-chain antibodies, ScFv) of such antibodies.

In one embodiment, the kit may further comprise instructions indicating that in the case of prH/E=the proportion of HER2-positive cells×100/(the proportion of ER-positive cells×100+1)≥1.5, the breast cancer is a breast cancer of an HER2-enriched-like subtype with a poor prognosis, wherein the proportions of the cells are proportions relative to the total number of tumor cells.

In one embodiment, the kit may further comprise a reagent for detecting the total number of tumor cells.

In one embodiment, the kit comprises a detection reagent that is only a molecule or compound for detecting the level of ERBB2 mRNA and the level of ESR1 mRNA expressed as FPKM, e.g., a primer set for ERBB2 and ESR1, and/or is only a molecule or compound for detecting HER2-positive cells and ER-positive cells, e.g., an antibody or an antigen-binding fragment thereof for the HER2 protein and the ER protein, and optionally a reagent for detecting the total number of tumor cells.

In one aspect, the disclosure provides a kit for predicting a breast cancer of an HER2-enriched subtype and/or an HER2-enriched-like subtype with a poor prognosis, comprising the detection reagent for detecting the level of ERBB2 mRNA and the level of ESR1 mRNA expressed as FPKM and/or the detection reagent capable of detecting HER2-positive cells and ER-positive cells described herein. The kit may further comprise any other suitable substance required for performing the detection, e.g., a reagent for detecting the total number of tumor cells.

In one embodiment, the kit comprises a detection reagent that is only a detection reagent for detecting ERBB2 mRNA and ESR1 mRNA expressed as FPKM and/or a detection reagent for detecting HER2-positive cells and ER-positive cells, e.g., an antibody or an antigen-binding fragment thereof for the HER2 protein and the ER protein. In one embodiment, the kit further comprises a reagent that can measure the total number of tumor cells to determine the total number of tumor cells in the sample.

In one embodiment, the kit may further comprise instructions indicating the calculation of rH/E=log₂(FPKM_{ERBB2}+1)/[log₂(FPKM_{ESR1}+1)+1], wherein the greater the rH/E (e.g., compared to rH/E for a known breast cancer of a non-HER2-enriched subtype), the more prone the subject to a breast cancer of an HER2-enriched subtype with a poor prognosis.

In one embodiment, the kit may further comprise a reference rH/E for a known breast cancer of a non-HER2-enriched subtype.

In one embodiment, the kit may further comprise instructions indicating that in the case of prH/E=the proportion of HER2-positive cells×100/(the proportion of ER-positive cells×100+1)≥1.5, the breast cancer is a breast cancer of an HER2-enriched-like subtype with a poor prognosis, wherein the proportions of the cells are proportions relative to the total number of tumor cells.

In one aspect, the disclosure provides a method for treating a subject with breast cancer, comprising measuring or obtaining the level of ERBB2 mRNA and the level of ESR1 mRNA expressed as FPKM and/or the number/proportion of HER2-positive cells and the number/proportion of ER-positive cells in a breast cancer tissue sample from the subject, optionally comprising measuring or obtaining the total number of cells in the sample (e.g., using the kit or apparatus described herein), and calculating the indicator rH/E described herein, wherein in the case of rH/E=log₂(FPKM_{ERBB2}+1)/[log₂(FPKM_{ESR1}+1)+1] being greater and/or prH/E=the proportion of HER2-positive cells×100/(the proportion of ER-positive cells×100+1)≥1.5 (wherein the proportions of the cells are proportions relative to the total number of tumor cells), the breast cancer is indicated to be a breast cancer of an HER2-enriched subtype and/or a breast cancer of an HER2-enriched-like subtype with a poor prognosis, and an intensive anti-HER2 therapy is recommended to be administered to the subject or an intensive anti-HER2 therapy is administered to the subject.

As used herein, the term “treatment” refers to alleviating at least one symptom of cancer. The term includes the application of one or more drugs to a subject to provide management or treatment of cancer. “Treatment” for the purposes of the present disclosure can, but does not have to, provide a cure; rather, “treatment” may be a form of management of the disorder. As used herein, “treatment” of a subject having cancer refers to the partial or complete elimination of the cancer in the subject, or to the maintenance of stability after treatment without further progression. The treatment includes prevention, treatment, and/or cure. Prevention refers to preventing the development of an underlying cancer and/or preventing the deterioration or progression of cancer. Preventing the development of cancer including the reduction or elimination of one or more risk factors that contribute to the development of cancer. When used herein to treat unwanted proliferating cells (including cancer), “treatment” includes the partial or complete destruction of the unwanted proliferating cells with minimal disruption to normal cells.

As used herein, the intensive anti-HER2 therapy refers to a therapy with an increased intensity (i.e., dual-target therapy with trastuzumab in combination with pertuzumab) or with an increased duration (i.e., sequential therapy with trastuzumab and neratinib therapy) on the basis of anti-HER2 therapy with trastuzumab.

In one embodiment, the biological sample described herein is a tumor tissue sample, preferably a breast cancer tissue sample, e.g., a tumor (breast cancer) tissue section.

In one aspect, the disclosure provides an apparatus comprising a digital processor, wherein the digital processor is configured to perform the method for predicting whether a breast cancer in a subject is a breast cancer of an HER2-enriched subtype and/or an HER2-enriched-like subtype with a poor prognosis or the method for treating a subject with breast cancer described herein.

In one aspect, the disclosure provides a non-transitory storage medium storing instructions, wherein the instructions are executable by a digital processing device to perform the method for predicting whether a breast cancer in a subject is a breast cancer of an HER2-enriched subtype and/or a breast cancer of an HER2-enriched-like subtype with a poor prognosis or the method for treating a subject with breast cancer described herein.

In one aspect, the disclosure provides a computer program comprising a program code module, wherein the program code module is configured to cause, when the computer program is run by a digital processing device, the digital processing device to perform the method for predicting whether a breast cancer in a subject is a breast cancer of an HER2-enriched subtype and/or a breast cancer of an HER2-enriched-like subtype with a poor prognosis or the method for treating a subject with breast cancer described herein.

In one aspect, the disclosure provides an apparatus for predicting a breast cancer of an HER2-enriched subtype with a poor prognosis, comprising:
a component for measuring or receiving the level of ERBB2 mRNA expressed as FPKM in a biological sample from a subject,
a component for measuring or receiving the level of ESR1 mRNA expressed as FPKM in the biological sample from the subject, and
a component for calculating an indicator rH/E=log₂(FPKM_{ERBB2}+1)/[log₂(FPKM_{ESR1}+1)+1];
optionally further comprising a component for displaying the calculation results for rH/E, wherein the greater the rH/E (e.g., compared to rH/E for a known breast cancer of a non-HER2-enriched subtype), the more prone the subject to an HER2-enriched subtype of breast cancer with a poor prognosis.

In one aspect, the disclosure provides an apparatus for predicting a breast cancer of an HER2-enriched-like subtype with a poor prognosis, comprising:
a component for measuring or receiving the number/proportion of HER2-positive cells in a biological sample from a subject, and
a component for measuring or receiving the number/proportion of ER-positive cells in the biological sample from the subject;
optionally comprising a component for measuring or receiving the total number of tumor cells in the biological sample from the subject, and
a component for calculating an indicator prH/E=the proportion of HER2-positive cells×100/(the proportion of ER-positive cells×100+1), wherein the proportions of the cells are proportions relative to the total number of tumor cells;
optionally comprising a component for indicating that the subject has a breast cancer of an HER2-enriched-like subtype with a poor prognosis in the case of prH/E≥1.5, e.g., a component giving a specific signal (such as sound and visual information), such as an electronic display screen.

In one embodiment, the biological sample is a tumor tissue sample, preferably a breast cancer tissue sample, e.g., a tumor (breast cancer) tissue section.

In one embodiment, the apparatus for predicting a breast cancer of an HER2-enriched subtype and/or HER2-enriched-like subtype with a poor prognosis comprises a digital processor configured to calculate the indicators rH/E and/or prH/E described herein.

The number or proportion of HER2− and ER-positive cells in the biological sample from the subject can be measured using the kit described herein, or can be obtained from other suitable measurement methods or means. The component for measuring or receiving the number/proportion of HER2-positive cells in the biological sample from the subject or the component for measuring or receiving the number/proportion of ER-positive cells in the biological sample from the subject may comprise materials and tools for measuring the number/proportion of HER2− and ER-positive cells in the biological sample (e.g., the detection reagent, kit, and the like described herein) or receive data from other sources regarding the number/proportion of HER2− and ER-positive cells.

In one embodiment, a non-transitory storage medium for calculating the rH/E and/or prH/E described herein stores instructions executable by a digital processing device to perform the method described herein. The non-transitory storage medium may be a computer-readable storage medium, such as a hard disk drive or other magnetic storage medium, an optical disk or other optical storage medium, random access memory (RAM), read only memory (ROM), flash memory, or other electronic storage medium, a network server, or the like. The digital processing device may be a portable device (e.g., a personal data assistant or smart phone), a laptop computer, a desktop computer, a tablet computer or device, a remote network server, or the like.

In one embodiment, a computer program for calculating the rH/E and/or prH/E described herein comprises a program code apparatus for causing, when the computer program is run by a digital processing device, the digital processing device to perform the method described herein. The digital processing device may be a portable device (e.g., a personal data assistant or smart phone), a laptop computer, a desktop computer, a tablet computer or device, a remote network server, or the like.

In one embodiment, the apparatus may be connected to a digital processing device (e.g., a personal data assistant or smart phone, a laptop computer, a desktop computer, a tablet computer or device, a remote network server, or the like) to provide relevant prompts to a patient or a person involved.

As used herein, a component for displaying measurement results may inform a subject or associated medical personnel of the measurement results (e.g., being greater than or less than, a particular numerical value, or the like) in any suitable manner (e.g., an alert tone, a digital signal, a networked message, or the like), including, for example, a display screen, or the like.

The apparatus may be interconnected with other systems, including but not limited to smartphones, tablet computers, laptop computers, and combinations of computing devices and cloud computing resources.

Embodiments of the apparatus and method described herein may be implemented in various systems including, but not limited to, smartphones, tablet computers, laptop computers, and combinations of computing devices and cloud computing resources. For example, some operations may occur in one device, while the other operations may occur at a remote location, such as one or more remote servers. For example, the collection of data may be performed at a smartphone and the data analysis may be performed at a server or cloud computing resource. Any single computing device or combination of computing devices may perform the method.

As used herein, “optionally present” or “optionally” means that the subsequently described event or circumstance occurs or does not occur, and that the description includes instances where the event or circumstance occurs and instances where it does not.

As used herein, the term “about” refers to a range of numerical values that includes a specific numerical value, which may reasonably be considered by those skilled in the art to be similar to the specific numerical value. In embodiments, the term “about” means within standard error of using measurements commonly accepted in the art. In certain embodiments, about refers to +/−10% or 5% of the specific numerical value.

### EXAMPLE

The disclosure is further illustrated by the following examples, but any examples or combination thereof should not be construed as limiting the scope or embodiment of the disclosure. Examples without specified techniques or conditions are implemented according to those described in literature in the art (e.g., see J. Sambrook et al., Molecular Cloning: A Laboratory Manual, third edition, Scientific Press, translated by Huang Peitang et al.) or according to product instructions.

### Example 1

The study included 3 cohorts of patients with HR+/HER2+ breast cancer. The first cohort included 141 patients from The Cancer Genome Atlas (TCGA). Clinical data for the TCGA cohort were extracted from the University of California Santa Cruz (UCSC) Xena (http://xena.ucsc.edu/).

The second cohort recruited 104 patients from Molecular Taxonomy of Breast Cancer International Consortium (METABRIC). Data for the METABRIC cohort were obtained from the European Bioinformatics Institute and deposited in the European Genome-Phenome Archive (http://www.ebi.ac.uk/ega/) under accession number EGAS00000000083.

The third cohort was a retrospective observational cohort of 43 patients with early HR+/HER2+ breast cancer treated at the Cancer Hospital of the Chinese Academy of Medical Sciences (CAMS) between 2012 and 2016. The CAMS cohort was recruited according to the following criteria: 1) female patients with stage I-III primary unilateral breast cancer who had undergone radical surgery and complete 1-year trastuzumab treatment; 2) patients with breast cancer being the first and only malignant cancer diagnosed; 3) all patients having formalin-fixed, paraffin-embedded surgical specimens, clinical data, and follow-up data; 4) patients having invasive ductal carcinoma with an HR+/HER2+ phenotype; and 5) approval by the Medical Ethics Committee of the Cancer Hospital of the CAMS. Exclusion criteria were as follows: 1) male patients, 2) bilateral primary breast cancer, 3) combined with other malignant tumors, and 4) no corresponding clinical or follow-up data.

ER, progestogen receptor (PR), and HER2 states were determined by IHC analysis or in situ hybridization. A cutoff value of ≥1% positive tumor cells was used to define ER and PR positivity, and HR positivity was defined as ER and/or PR positivity. The HER2 state was defined according to the most recent ASCO/CAP guidelines. Follow-up was completed on May 8, 2020, with a median follow-up duration of 64 months (interquartile range, 20-101 months). The study was approved by the independent ethics committee/institutional review board of the CAMS (20/272-2468).

### Example 2: Bioinformatic Analysis

### Somatic Mutation Analysis

Somatic mutation data for the TCGA cohort were extracted from UCSC Xena (http://xena.ucsc.edu/), and the most commonly mutated cancer-related genes in HER2+ breast cancer were identified. Differences in somatic mutations between HER2-enriched and non-HER2-enriched subtypes (Liminal A, liminal B, basal cell-like, and normal breast-like) were compared using the “maftools” R software package [Mayakonda A et al., (2018) Genome Res 28: 1747-1756. doi: 10.1101/gr.239244.118].

### RNA-Seq Analysis

RNA-seq data (level 3) for the TCGA cohort were extracted from UCSC Xena (http://xena.ucsc.edu/), and normalized gene expression was measured as fragments per kilobase of transcript per million mapped reads (FPKM). After adding a constant value of 1 to all values, the FPKM values were log 2-transformed. Genes that were statistically significantly differentially expressed were detected using the “limma” R software package. The “genefu” R software package was used to perform PAM50 typing on each patient [Parker J S et al., (2009) J Clin Oncol 27: 1160-1167. doi:10.1200/JCO.2008.18.1370].

Multiplex Immunofluorescence (mIF)

mIF was used to detect the expression of ER and HER2: red fluorescence in the cell nucleus indicated ER positivity and green fluorescence in the cell membrane indicated HER2 positivity. The number and proportion of different types of tumor cells were analyzed using Image-Pro Plus (Version 7.0.1.658, Media Cybernetics, Rockville, MD, USA) image processing and analysis software.

Multiplex IF staining techniques were used to visualize the expression of ER and HER2 at single cell resolution. First, tissue sections of the patients were deparaffinized and boiled in a tissue repair solution (Thermo, MA, USA) under high pressure and microwave irradiation for 15 min. The sections were then cooled to 20° C., treated with a blocking solution (Thermo) for >30 min, and incubated at 4° C. overnight with a diluted HER2 primary antibody (1:400, CST, MA, USA). Next, the sections were treated with a horseradish peroxidase (HRP)-conjugated secondary antibody (Thermo) for 2 h at 20° C. and then with a chromogenic solution (FITC 488, Thermo) for 6 min. After color development, the sections were washed 3 times with phosphate buffered saline (PBS) for 5 min on a shaker, boiled in a tissue repair solution (Thermo) under high pressure for 15 min, and cooled to 20° C. The sections were then treated with a blocking solution (Thermo) at 20° C. for >30 min, incubated with an ER primary antibody (1:200, CST) at 4° C. overnight, and treated with an HRP-conjugated secondary antibody (Thermo) at 20° C. for 2 h. After incubation with a chromogenic solution (Alexa 594, Thermo) for 6 min, the sections were washed 3 times with PBS on a shaker for 5 min and sealed with a neutral resin containing DAPI. The stained sections were then observed using a confocal microscope (Olympus, Tokyo, Japan) within 1 week.

The different colors of fluorescence correspond to different excitation wavelengths. In this study, ER positivity was indicated by red fluorescence in the cell nucleus, while HER2 positivity was indicated by green fluorescence in the cell membrane. To quantify the proportions of ER+HER2+, ER+HER2−, ER-HER2+, and ER-HER2− tumor cells, we randomly selected 10 high magnification fields of view (400×) (>100 tumor cells per field of view) in each of the tumors for processing and analysis using Image-Pro Plus (IPP) software. The average was used for statistical analysis.

### Statistical Analysis

Statistical analysis was performed using SPSS version 24.0 (SPSS Inc., Chicago, IL, USA), GraphPad Prism (Version 8.0, GraphPad Software Inc., La Jolla, CA, USA), X-tile Software (Version 3.6.1, Yale University School of Medicine, New Haven, CT, USA), and R Software (Version 3.6.0). Quantitative data with a normal distribution were described using the mean and standard deviation, with a t-test used for between-group comparisons, whereas quantitative data with a skewed distribution were described using the median and interquartile range, with a rank sum test used for between-group comparisons. Categorical variables were described using the number and percentage, and between-group comparisons were performed using a Pearson's chi-square test. Data with a normal distribution were analyzed using Pearson's correlation and data with a non-normal distribution were analyzed using Spearman's correlation. Disease-free survival (DFS) was used as an indicator of prognostic evaluation, defined as the time from surgery to first local, regional, or distant tumor recurrence or death. The optimal cut-off value of prH/E was determined using X-tile software and the DFS difference between the two groups was compared using Kaplan-Meier survival analysis. Univariate and multivariate Cox regression analyses were performed to determine important prognostic factors, and the performance of the prognostic model was evaluated by calculating Harrell's concordance index [Harrell F E Jr et al., (1996) Stat Med 15: 361-387. doi: 10.1002/(SICI)1097-0258(19960229)15:4<361::AID-SIM168>3.0.CO; 2-4]. P<0.05 was considered to indicate statistical significance.

### Results

### Distribution of PAM50 Intrinsic Subtypes of HR+/HER2+ Breast Cancer

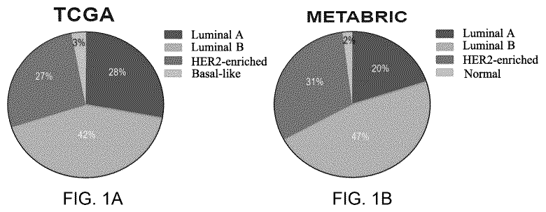

Both the TCGA and METABRIC datasets contained four PAM50 intrinsic subtypes. The liminal B subtype was the most common (42% (FIG. 1A); 47% (FIG. 1B)), and the HER2-enriched subtype (27% (FIG. 1A); 31% (FIG. 1B)) and the liminal A subtype (28% (FIG. 1A); 20% (FIG. 1B)) accounted for approximately one third of the two data sets. In the TCGA and METABRIC datasets, only a very few patients were classified as basal-like or normal subtypes, respectively (3% (FIG. 1A); 2% (FIG. 1B)).

### Comparison of Molecular Characteristics of HER2-Enriched and Non-HER2-Enriched Subtypes in HR+/HER2+ Breast Cancer

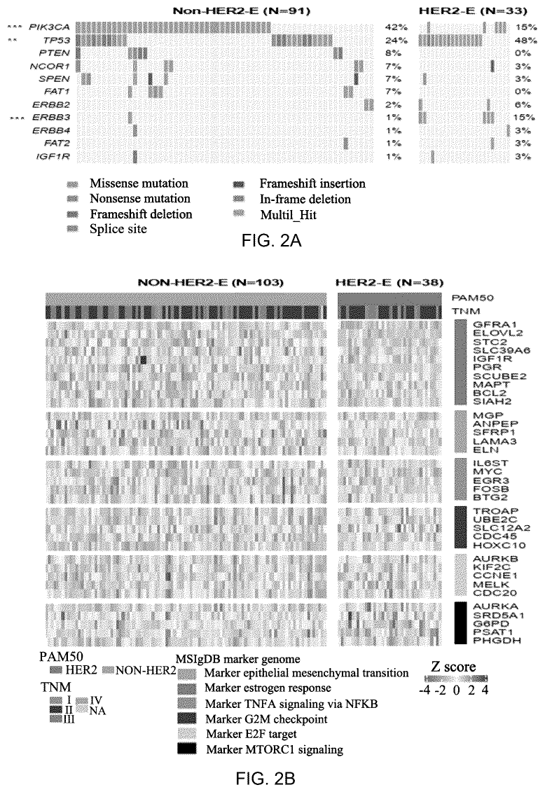

To better understand the inter-tumor heterogeneity, we determined the differences between the HER2-enriched and non-HER2-enriched subtypes by analyzing somatic mutation and RNA expression data. The HER2-enriched subtype was characterized by significantly higher mutation frequencies of TP53 (48% vs. 24%, p<0.01) and ERBB3 (15% vs. 1%, p<0.001) and a significantly lower mutation frequency of PIK3CA (15% vs. 42%, p<0.001) than the non-HER2-enriched subtype. (FIG. 2A)

Furthermore, the RNA-seq data for TCGA showed that: the expression of genes related to a G2/M cell cycle checkpoint, an E2F pathway, and a rapamycin complex 1 signaling pathway was significantly higher in the HER2-enriched subtype, while the expression of genes related to epithelial mesenchymal transition, estrogen response, and a tumor necrosis factor-α signaling pathway mediated by NF-κB was significantly lower, than in the non-HER2-enriched subtype. (FIG. 2B)

rH/E Better Predicted the HER2-Enriched Subtype

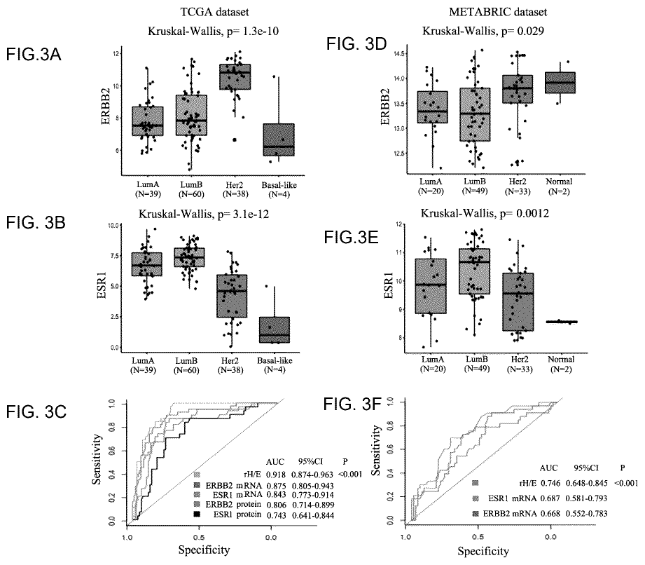

In the TCGA and METABRIC datasets, the HER2-enriched subtype was characterized by significantly higher ERBB2 mRNA expression and reduced ESR1 mRNA expression than the non-HER2-enriched subtype (FIGS. 3A, 3B, 3D, 3E). However, the HER2-enriched subtype and the non-HER2-enriched subtype cannot be readily distinguished by the expression of ERBB2 or ESR1 (FIGS. 3C, 3F).

Based on the above results, we constructed a novel marker, called rH/E, which was calculated as follows: ERBB2 expression quantity/(ESR1 expression quantity+1), wherein the expression quantity was expressed by Log₂(FPKM+1). Thus, rH/E reflected the relative expression of ERBB2 to ESR1 in each patient. To determine the best predictor for the HER2-enriched subtype, we compared area under the curve (AUC) values for ERBB2 expression, ESR1 expression, and rH/E. rH/E had the highest AUC value in the TCGA (AUC=0.918, 95% confidence interval [CI]: 0.874-0.963) and METABRIC (AUC=0.746, 95% CI: 0.648-0.845) datasets (FIGS. 3C, F).

### The Existence of 4 Tumor Cell Subtypes Reflected the Intra-Tumor Heterogeneity in HR+/HER2+ Breast Cancer

To further evaluate tumor heterogeneity in HR+/HER2+ breast cancer, we tested the expression of HER2 and ER proteins simultaneously using mIF in 43 patients with HR+/HER2+ breast cancer who had undergone surgery followed by chemotherapy and a 1-year adjuvant trastuzumab therapy in CAMS (CAMS cohort).

Characteristic features of the CAMS cohort are shown in Table 1. Nineteen patients had lymph node metastasis. Only 2 patients did not receive adjuvant endocrine therapy. At the final follow-up, 6 patients experienced tumor recurrence or progression. No significant difference in clinical pathology was detected between patients with and without recurrence.

TABLE 1
clinical pathology of 43 patients with double-positive breast cancer

| | | | | | No | | p |
|---|---|---|---|---|---|---|---|
| Variable | Total | | Recurrence | | recurrence | | value |
| Age at diagnosis | | | | | | | 0.705 |
| ≤50 years old | 22 | (51.16%) | 4 | (66.67%) | 18 | (48.65%) | |
| >50 years old | 21 | (48.84%) | 2 | (33.33%) | 19 | (51.35%) | |
| Lymph node metastasis | | | | | | | 0.452 |
| Negative | 24 | (55.81%) | 2 | (33.33%) | 22 | (59.46%) | |
| Positive | 19 | (44.19%) | 4 | (66.67%) | 15 | (40.54%) | |
| Grading | | | | | | | 1 |
| Grade II | 27 | (62.79%) | 4 | (66.67%) | 23 | (62.16%) | |
| Grade III | 16 | (37.21%) | 2 | (33.33%) | 14 | (37.84%) | |
| Staging | | | | | | | 0.178 |
| Stages I-II | 34 | (79.07%) | 3 | (50.00%) | 31 | (83.78%) | |
| Stage III | 9 | (20.93%) | 3 | (50.00%) | 6 | (16.22%) | |
| Surgical method | | | | | | | 0.864 |
| Breast | 12 | (27.91%) | 1 | (16.67%) | 11 | (29.73%) | |
| conserving | | | | | | | |
| surgery | | | | | | | |
| Others | 31 | (72.09%) | 5 | (83.33%) | 26 | (70.27%) | |
| Adjuvant chemotherapy | | | | | | | 0.524 |
| Without | | | | | | | |
| anthracycline | 19 | (44.19%) | 4 | (66.67%) | 15 | (40.54%) | |
| antibiotics | | | | | | | |
| With | 22 | (51.16%) | 2 | (33.33%) | 20 | (54.05%) | |
| anthracycline | | | | | | | |
| antibiotics | | | | | | | |
| No adjuvant | 2 | (4.65%) | | | 2 | (5.41%) | |
| chemotherapy | | | | | | | |
| Whether to receive tumor adjuvant therapy | | | | | | | 0.888 |
| Yes | 3 | (6.98%) | 1 | (16.67%) | 2 | (5.41%) | |
| No | 40 | (93.02%) | 5 | (83.33%) | 35 | (94.59%) | |
| Whether to receive targeted therapy | | | | | | | NS |
| Yes | 43 | (100%) | 6 | (100%) | 37 | (100%) | |
| No | 0 | (0) | 0 | (0) | 0 | (0%) | |
| Whether to receive endocrine therapy | | | | | | | 1 |
| Yes | 41 | (95.34%) | 6 | (100%) | 35 | (94.59%) | |
| No | 2 | (4.65%) | 0 | (0) | 2 | (5.41%) | |
| Whether to receive radiotherapy | | | | | | | 1 |
| Yes | 21 | (48.84%) | 3 | (50.00%) | 18 | (48.65%) | |
| No | 22 | (51.16%) | 3 | (50.00%) | 19 | (51.35%) | |

NS: not shown.

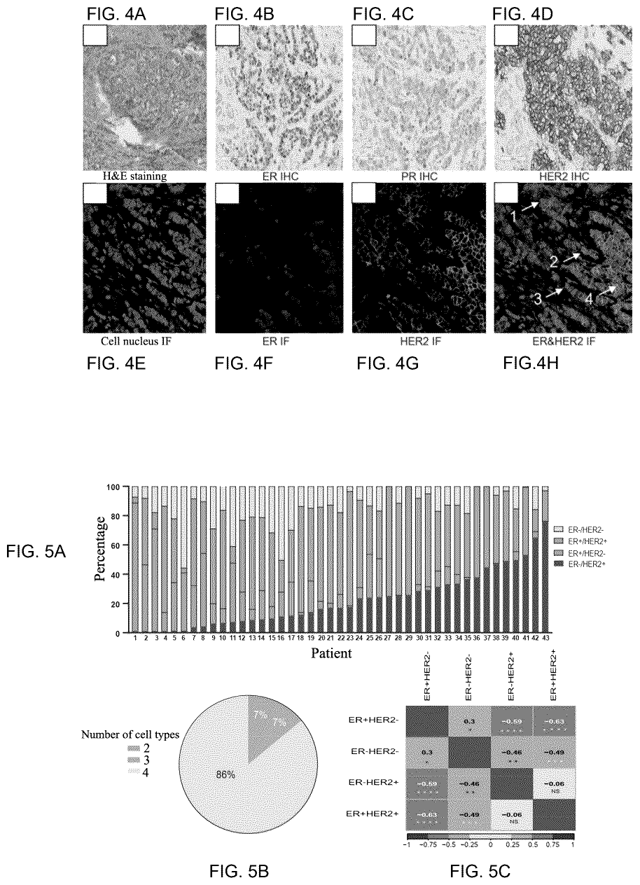

Interestingly, mIF showed that tumor cells of the HR+/HER2+ breast cancer can be divided into 4 classes based on the expression of HER2 and ER: ER+HER2+, ER+HER2−, ER-HER2+, and ER-HER2−. FIGS. 4A-4H shows mIF images of a patient with ER 80%+, PR 10%, and HER2 3+ breast cancer.

In addition, we found that the distribution of 4 tumor cell subtypes was distinct among patients. A total of 7%, 7%, and 86% of patients had 2, 3, and 4 tumor cell types, respectively (FIG. 5B). The proportion of ER-HER2+ tumor cells was correlated negatively with the proportion of ER+HER2− and ER-HER2− tumor cells, and the proportion of ER-HER2− tumor cells was correlated positively with the proportion of ER+HER2− tumor cells (FIGS. 5A, 5C).

Potential Clinical Significance of prH/E in CAMS Cohort

When using mIF, rH/E was adjusted to prH/E, which was calculated as follows:

the percentage of HER2-positive cells(ER+HER2+ and ER-HER2+)×100/(the percentage of ER-positive cells[ER+HER2+ and ER+HER2−]×100+1).

In the CAMS cohort, we evaluated the clinical correlation of these 4 tumor cell types. Interestingly, prH/E, but not the tumor cell phenotype, showed a significant prognostic correlation. TNM staging and prH/E were independent risk factors for DFS (Table 2).

TABLE 2
univariate and multivariate Cox regression analyses

| | Univariate Cox | | | Multivariate | | |
|---|---|---|---|---|---|---|
| | regression | | | Cox regression | | |
| Variable | HR | 95% CI | p value | HR | 95% CI | p value |
| TNM | 3.83 | 1.04-14.05 | 0.04 | 4.60 | 1.29-16.38 | 0.02 |
| Age | 0.93 | 0.85-1.02 | 0.14 | | | |
| ER+HER2− | 0.96 | 0.90-1.04 | 0.33 | | | |
| ER−HER2+ | 1.04 | 1.00-1.08 | 0.07 | | | |
| ER+HER2+ | 1.00 | 0.96-1.04 | 0.96 | | | |
| ER−HER2− | 0.96 | 0.88-1.05 | 0.37 | | | |
| prH/E | 2.09 | 1.04-4.21 | 0.04 | 2.63 | 1.15-6.01 | 0.02 |

Abbreviations: HER2: human epidermal growth factor receptor 2; ER: estrogen receptor; HR: hormone receptor or risk ratio; CI: confidence interval

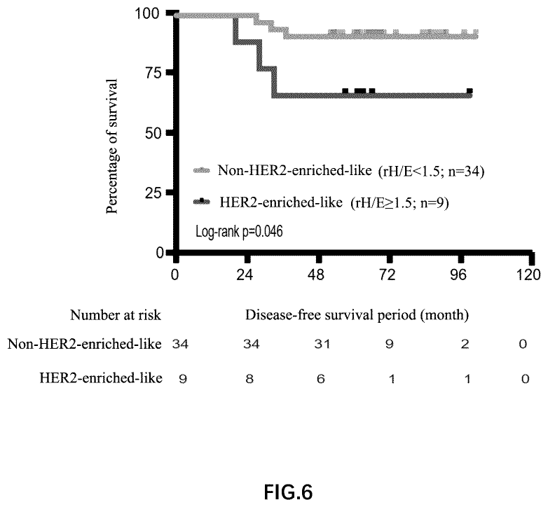

The combination of prH/E and TNM staging significantly increased prognostic predictive efficacy compared to TNM staging alone (Table 3). The X-tile software determined the optimal cut-off value of prH/E at 1.5. According to the level of prH/E, we divided the 43 patients into an HER2-enriched-like subgroup (n=9) and a non-HER2-enriched-like subgroup (n=34). The HER2-enriched-like subgroup with higher prH/E showed significantly reduced 5-year DFS than the non-HER2-enriched-like subgroup (67% vs. 91%, log-rank p=0.046) (FIG. 6).

TABLE 3
prognostic predictive efficacy of
TNM staging with or without rH/E

| Variable | C-index | 95% CI | p value |
|---|---|---|---|
| TNM | 0.74 | 0.60-0.89 | 0.022 |
| TNM and prH/E | 0.80 | 0.63-0.96 | |

Abbreviations:
CI: confidence interval

1. Use of a detection reagent for detecting ERBB2 mRNA expressed as fragments per kilobase of transcript per million mapped reads (FPKM) and a detection reagent for detecting ESR1 mRNA expressed as FPKM and/or a detection reagent for detecting HER2− positive cells and a detection reagent for detecting ER-positive cells in the preparation of a kit for predicting a breast cancer of an HER2-enriched subtype and/or a breast cancer of an HER2− enriched-like subtype with a poor prognosis.

2. The use according to claim 1, wherein the detection reagent is a detection reagent, e.g., an antibody or an antigen-binding fragment thereof, that specifically binds to an HER2 protein or an ER protein, and optionally, the kit comprises instructions indicating that in the case of prH/E=the proportion of HER2-positive cells×100/(the proportion of ER-positive cells×100+1)≥1.5, the breast cancer is a breast cancer of an HER2-enriched-like subtype with a poor prognosis.

3. A kit for predicting a breast cancer of an HER2-enriched-like subtype with a poor prognosis, comprising a detection reagent for detecting HER2-positive cells and a detection reagent for detecting ER-positive cells, wherein preferably, the detection reagent is a detection reagent, e.g., an antibody or an antigen-binding fragment thereof, that specifically binds to an HER2 protein or an ER protein, and optionally, the kit comprises instructions indicating that in the case of prH/E=the proportion of HER2-positive cells×100/(the proportion of ER-positive cells×100+1)≥1.5, the breast cancer is a breast cancer of an HER2-enriched-like subtype with a poor prognosis, wherein the proportion of the cells are proportions relative to the total number of tumor cells.

4. An apparatus for predicting a breast cancer of an HER2-enriched-like subtype with a poor prognosis, comprising:
a component for measuring or receiving the number/proportion of HER2-positive cells in a biological sample from a subject,
a component for measuring or receiving the number/proportion of ER-positive cells in the biological sample;
optionally comprising a component for measuring or receiving the total number of tumor cells in the biological sample, and
a component for calculating prH/E=the proportion of HER2-positive cells×100/(the proportion of ER-positive cells×100+1), wherein the proportions of the cells are proportions relative to the total number of tumor cells;
optionally comprising a component for indicating that the subject has a breast cancer of an HER2-enriched-like subtype with a poor prognosis and/or recommending administering an intensive anti-HER2 therapy to the subject in the case of rH/E≥1.5.

5. The apparatus according to claim 4, wherein the biological sample is a breast cancer tissue sample, e.g., a breast cancer tissue section.

6. A method for predicting whether a breast cancer in a subject is a breast cancer of an HER2-enriched-like subtype with a poor prognosis, comprising:
measuring or obtaining the number/proportion of HER2-positive cells in a biological sample from a subject,
measuring or obtaining the number/proportion of ER-positive cells in the biological sample;
optionally comprising measuring or obtaining the total number of tumor cells in the biological sample, and
calculating prH/E=the proportion of HER2-positive cells×100/(the proportion of ER-positive cells×100+1), wherein the proportions of the cells are proportions relative to the total number of tumor cells,
wherein in the case of prH/E≥1.5, the breast cancer in the subject is indicated to be a breast cancer of an HER2-enriched-like subtype with a poor prognosis.

7. A method for treating a subject with breast cancer, comprising measuring or obtaining the number/proportion of HER2-positive cells and the number/proportion of ER-positive cells in a biological sample of the subject, wherein in the case of prH/E=the proportion of HER2-positive cells×100/(the proportion of ER-positive cells×100+1)≥1.5, an intensive anti-HER2 therapy is recommended to be administered the subject or an intensive anti-HER2 therapy is administered to the subject, wherein the proportions of the cells are proportions relative to the total number of tumor cells.

8. An apparatus comprising a digital processor, wherein the digital processor is configured to perform the method according to claim 6.

9. A non-transitory storage medium storing instructions, wherein the instructions are executable by a digital processing device to perform the method according to claim 6.

10. A computer program comprising a program code module, wherein the program code module is configured to cause, when the computer program is run by a digital processing device, the digital processing device to perform the method according to claim 6.
